(19) United States
(12) Patent Application Publication
(10) Pub. No.: US 2015/0217002 A1
(43) Pub. Date: Aug. 6, 2015

(54) NEAR-INFRARED SPECTROSCOPY AND OPTICAL REPORTER

(71) Applicant: THE RESEARCH FOUNDATION FOR THE STATE UNIVERSITY OF NEW YORK, Albany, NY (US)

(72) Inventors: Lilianne R. Mujica-Parodi, Stony Brook, NY (US); Balaji Sitharaman, Coram, NY (US); Daniel Dedora, Greenlawn, NY (US); Sayan Mullick Chowdhury, Stony Brook, NY (US)

(73) Assignee: THE RESEARCH FOUNDATION FOR THE STATE UNIVERSITY OF NEW YORK, Albany, NY (US)

(21) Appl. No.: 14/420,124

(22) PCT Filed: Aug. 9, 2013
(86) PCT No.: PCT/US2013/054343

§ 371 Date: Feb. 6, 2015

Related U.S. Application Data
(60) Provisional application No. 61/681,723, filed on Aug. 10, 2012.

(51) Int. Cl. A61K 49/00; A61B 5/145; A61B 5/00; A61B 5/1455

(52) U.S. Cl. CPC A61K 49/0063; A61B 5/1455; A61B 5/14532; A61B 5/14546; A61B 5/0013; A61B 2560/0412; A61B 2562/0238; A61B 2560/0443; A61B 2576/00

(57) ABSTRACT

The method of the present disclosure is directed towards a method for determining the concentration of an analyte in a mammal's bloodstream. This method includes the following steps; applying a patch to the mammal's skin, the patch configured to transmit a reporter through the mammal's skin, wherein the reporter is configured to have an affinity for the analyte and is capable of exhibiting a detectable change in fluorescence upon binding to the analyte, subjecting a portion of the mammal's skin to Near Infrared Spectroscopy (NIRS), detecting fluorescence emission intensity and calculating the concentration of the analyte based on the detected fluorescence emission. The system of the present disclosure is directed towards an analyte monitoring system. The system includes the following, a patch and a receiver

### CROSS REFERENCE TO RELATED APPLICATIONS

This application claims the benefit of U.S. Provisional Application No. 61/681,723 filed on Aug. 10, 2012.

### STATEMENT REGARDING FEDERALLY SPONSORED RESEARCH OR DEVELOPMENT

This invention was made with government support under grant number CBET0954643 awarded by the National Science Foundation. The government has certain rights in the invention.

### BACKGROUND OF THE DISCLOSURE

Monitoring of the levels of various substituents in the blood, such as glucose, is inefficient in several respects. Samples of blood must be manually taken, which is painful and requires sanitary conditions and devices. Further, each individual sample only offers a point in time reference of the actual substituent, without the measurement of whether the concentration is increasing or decreasing and at what rates this increase or decrease is occurring.

For example, long-term measurement of glucose in a person's blood traditionally has several drawbacks for individuals that have diabetes. Diabetes is a metabolic dysregulatory disorder that affects about 25 million Americans and about 200 million people worldwide. The current standard of care for glucose management is the finger-prick assay. Each measurement requires a fresh blood sample; this results in low patient compliance, especially among children and elderly patients who have difficulty in obtaining samples. Furthermore, isolated measurements give little information about the unstable temporal dynamics of glucose in the body, which limits their predictive utility and precludes closed-circuit insulin pump control.

As can be seen in FIG. 1, sporadic finger-prick readings do not provide a good indication of the actual increases and decreases in glucose levels a subject goes through each day. These sporadic finger-prick readings are static snapshots of what the person's glucose levels go through each day and do not provide a good idea of the dynamics of the glucose levels.

What is desired is a method and system for determining an analyte in a mammal's bloodstream that is non-invasive and is accurate. Embodiments of the present disclosure provide a method and system that addresses the above and other issues.

### SUMMARY OF THE DISCLOSURE

The present disclosure is directed to a patch to be applied to a mammal's skin to deliver a reporter and detect a resultant fluorescence.

A method of the present disclosure is directed towards a method for determining the concentration of an analyte in a mammal's bloodstream. This method includes the following steps: 1) applying a patch to the mammal's skin, the patch configured to transmit a reporter through the mammal's skin, wherein the reporter is configured to have an affinity for the analyte and is capable of undergoing a detectable change in fluorescence upon binding to the analyte, 2) subjecting a portion of the mammal's skin to Near Infrared Spectroscopy (NIRS), 3) detecting fluorescence emission intensity, and 4) calculating the concentration of the analyte based on the detected shift in fluorescence.

The present disclosure is further directed to an analyte monitoring system. The system includes the following, a patch and a receiver/analysis device.

### BRIEF DESCRIPTION OF THE DRAWINGS

The present disclosure will be better understood by reference to the following drawings of which:

FIG. 1 is a graphical representation of glucose readings gathered according to a finger prick method;

FIG. 2 is a graphical representation of the patch;

FIG. 3 is a graphical representation of the patch;

FIG. 4 is a graphical representation of the reporter;

FIG. 5a and FIG. 5b are two sample NIR spectra of the reporter with and without glucose;

FIG. 6 is a sample Raman spectra of the reporter with and without glucose;

FIG. 7 is a graphical representation of a separated delivery and detection system;

FIG. 8 is a series of in situ transdermal permeation tests in pig skin;

FIGS. 9a-9c are graphical illustrations of NIR absorbance of various compounds;

FIGS. 10a-10c are graphical illustrations of the fluorescence of various compounds; and

FIGS. 11a-11c are graphical illustrations of the fluorescence of various compounds.

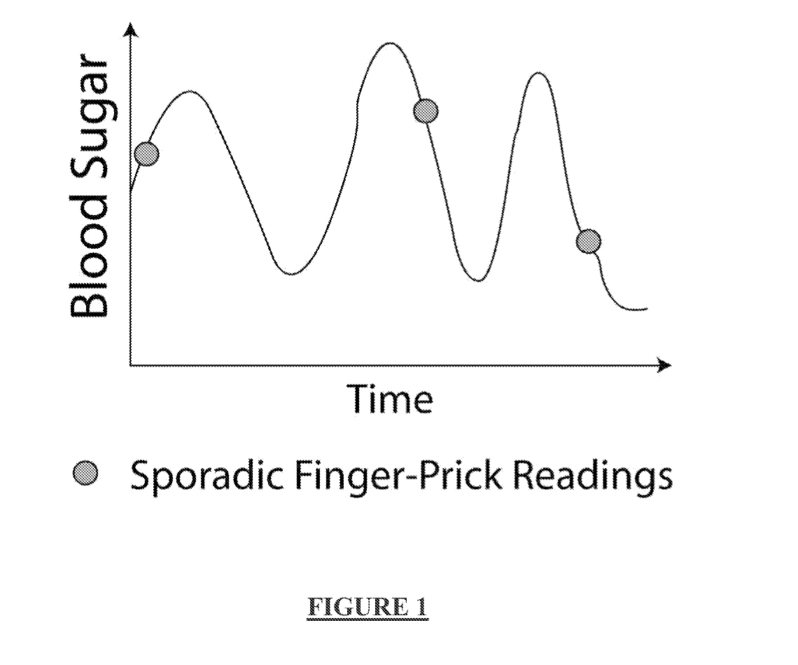

### DETAILED DESCRIPTION OF THE DISCLOSURE

In the claimed method and system, a patch is first applied to a mammal's skin. In other embodiments, a continuous source of a reporter can be used, such as an intravenous drip or intravenous injection. This patch can be placed in any suitable area of the mammal's skin that can accept a reporter and emit fluorescence. In one embodiment, the patch is placed over a superficial capillary bed, which is approximately one centimeter from the surface of the skin. The patch is configured to transmit a reporter through the mammal's skin; the patch may operate through passive diffusion, diffusion with a chemical permeation enhancer such as oleic acid, diffusion with a physical permeation enhancer such as heat or ultrasound, or diffusion with a microneedle array for bypassing of the stratum corneum barrier. The reporter is configured to have an affinity for an analyte. The analyte can be any material in the blood stream, including but not limited to glucose, blood oxygen levels and hormones such as cortisol, testosterone, and other blood analytes of interest.

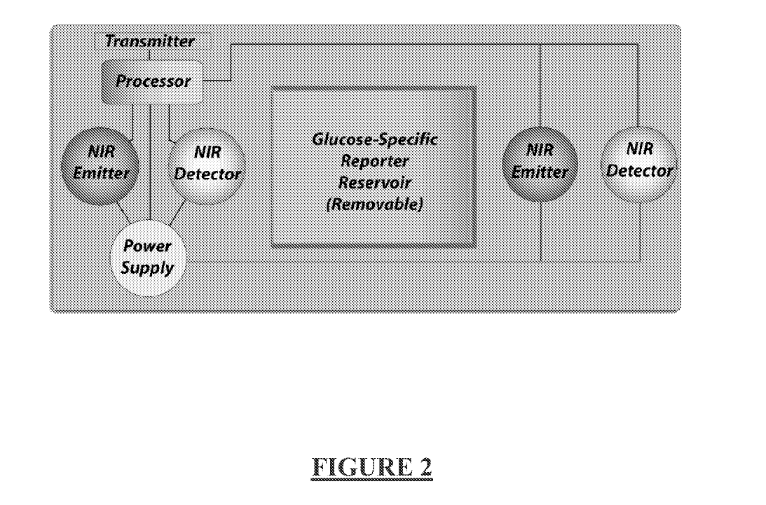

A graphical representation of the patch can be seen in FIG. 2, a patch 2 will be used that includes a backing layer (not shown), a reporter matrix 3 and a liner (not shown), the patch able to release a near constant dosage of reporter for extended periods of up to seven days with minimal, if any, skin irritation. The patch 2 can be any suitable patch capable of transmitting a reporter through a mammal's skin for a period of time. This period of time can be from about 1 minute to about one month or longer depending on the reporter to be delivered. The patch 2 can also include an adhesive to keep the patch in contact with the mammal's skin.

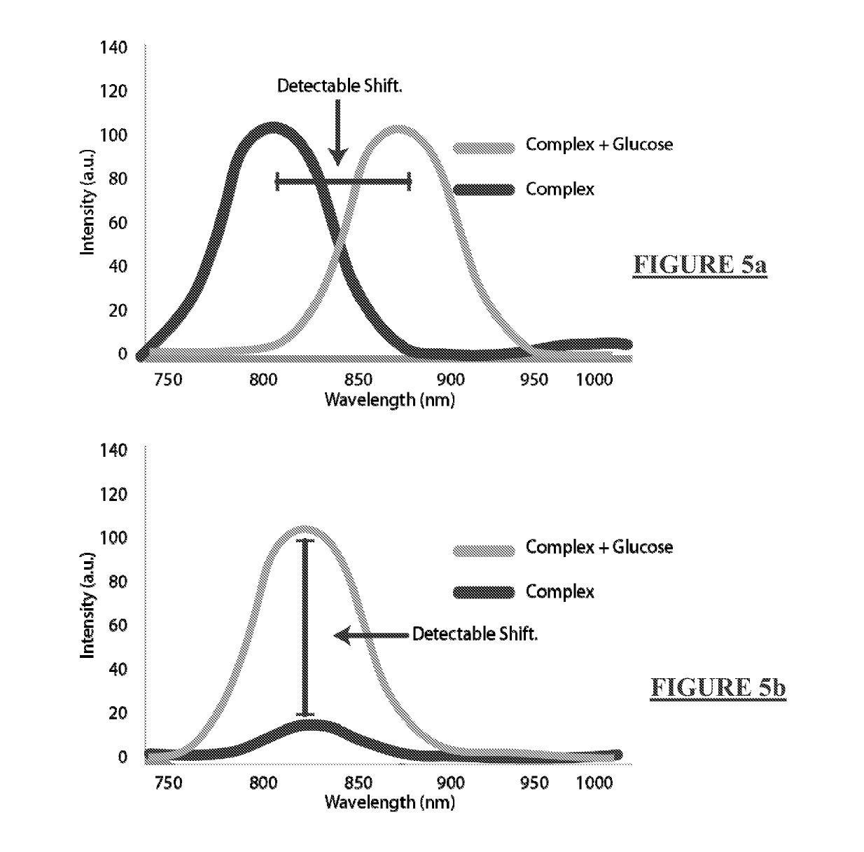

Also included in the patch 2 is a Near Infrared (NIR) emitter 4 and detector 6 capable of detecting NIR signals. Although FIG. 2 indicates that there is one emitter 4 and one detector 6, a plurality of emitters and detectors can be included. Additionally, the emitter and detector may be two or more separate portions, as shown in FIG. 5 and as described below. The emitter 4 and detector 6 are electronically integrated with a processor 8, transmitter 10 and a power supply 12. The processor 8 can be any suitable processor configured to receive and process at least one detected light signal to produce a measured level output to be transmitted by transmitter 10. Transmitter 10 is further discussed below. The power supply 12 can be any suitable supply, including but not limited to a battery.

In the reporter matrix 3, the reporter can be any optical reporter that is a fluorophore and binds with a specific analyte and is capable of being absorbed through the dermal layers of a mammal's skin. In one example, a glucose-specific optical reporter in which the binding site of a boronic acid derivative is co-ordinated to an Indocyanine green (ICG)-labeled cyclodextrin, a cyclic ring sugar molecule, serves as the reporter. In other embodiments, the reporter can be another ICG complex, such as an alpha, beta, or gamma cyclodextrins, as well as their derivatives, such as hydroxypropyl and methyl beta cyclodextrin, as well as ICG cyclodextrin covalent complex, co-ordinated with a boronic acid derivative.

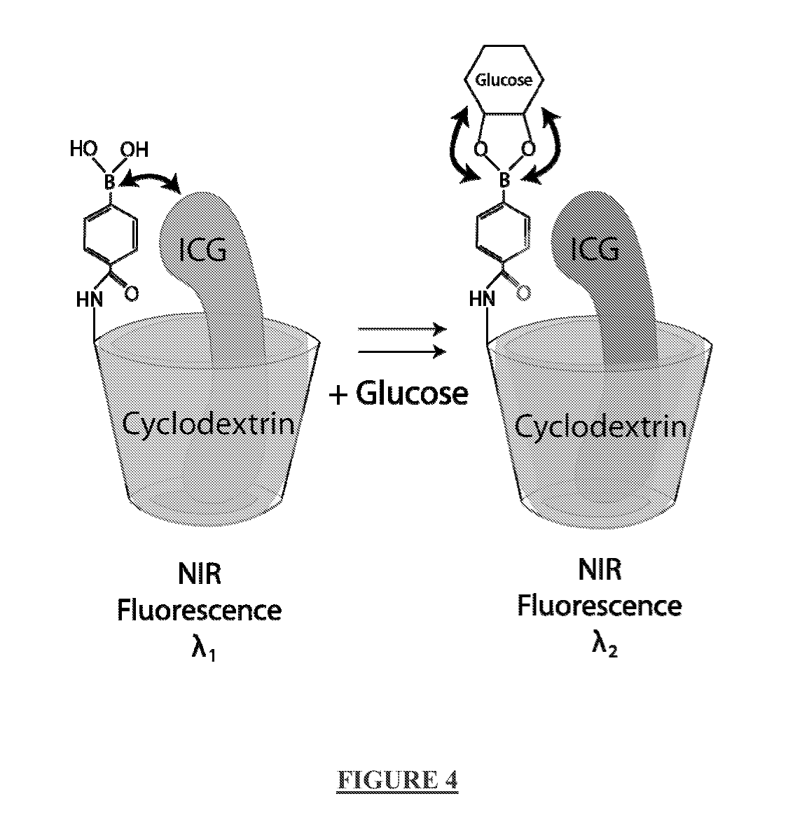

A graphical representation of the optical reporter boronic acid derivative co-ordinated to an ICG-labeled cyclodextrin is provided in FIG. 4. As graphically shown in FIG. 4, upon addition of glucose to boronic acid ICG-labeled cyclodextrin, the glucose binds to the boronic acid, causing the ICG-labeled cyclodextrin to fluoresce at a different wavelength than unbound reporter. In one embodiment of the application, this change in wavelength between bound analyte and unbound optical reporter is shown in FIG. 5a. Regarding FIGS. 5a and 5b, the analyte detected in these figures was, as one example of possible analytes, glucose. The difference in wavelength between bound and unbound reporters is detectable, and the ratio between unbound and bound fluorescence can be used to calculate analyte concentration. The equilibrium equation is:

k=[reporter][analyte]/[reporter+analyte]

wherein k is an empirically derived binding constant between the reporter complex and analyte in blood, and [reporter] and [reporter+analyte] is a ratio acquired from measuring fluorescence at the two wavelengths, representing the unbound and bound states of the reporter. Solving for [analyte] will result in measures of intra-vessel analyte concentrations.

In another embodiment of the application, the reporter exhibits a modulation in fluorescence upon binding to an analyte as shown in FIG. 5b. In the example illustrated in FIG. 5b, the fluorescence is increased upon binding with glucose, in other examples, the fluorescence may decrease upon binding with glucose or any other suitable analyte. In this example, an increase in fluorescence is caused by an analyte-induced conformational change in the reporter molecule; this conformational change reduces the fluorescence resonance energy transfer (FRET) between fluorophore (donor) and FRET acceptor moiety.

The change in wavelength, the change in fluorescence intensity or the change in wavelength and fluorescence intensity would then be correlated with the concentration of analyte in the blood, and a patient specific correlation equation would be solved to measure analyte concentration.

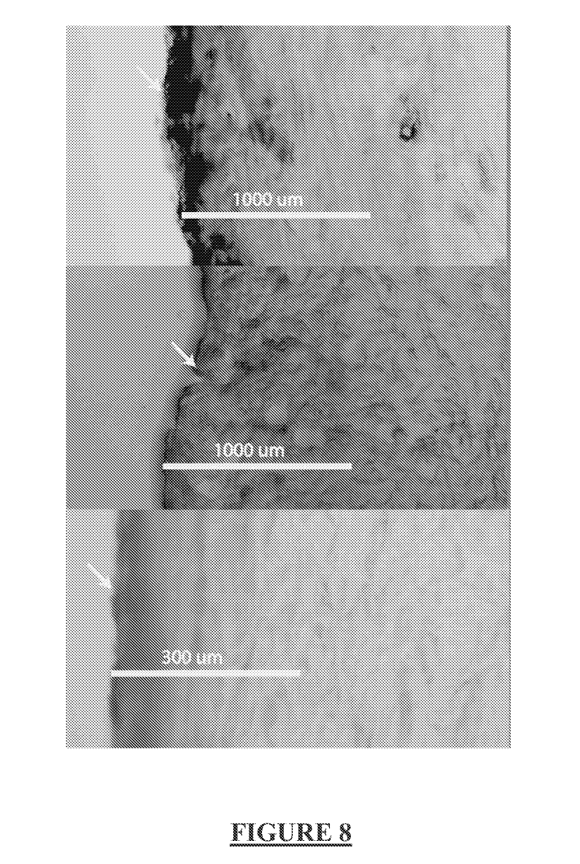

A sample optical reporter shown above and in FIG. 4 has a molecular weight of approximately 2400 Da, and is soluble in both water and alcohol. As shown in FIG. 8, this reporter is able to permeate through the dermal layers of a mammal's skin.

Referring to the reporter in the reporter matrix 3, in this example, indocyanine green (ICG) is a cyanine dye that has a peak spectral absorption at about 800 nm. ICG has an in vivo half-life of about 3 minutes and is removed from the circulation system by the liver. ICG is a Food and Drug Administration (FDA)-approved non-specific fluorophore with a low molecular weight of about 775 Da.

In this example of the reporter in reporter matrix 3, any boronic acid derivative that is capable of glucose binding can also be used with the reporter. As another example, any molecule or moiety that is specific for an analyte of interest may be used as the reporter. As one example of the reporter in reporter matrix 3, a cyclodextrin can be used, the cyclodextrin can be any molecule arranged as a ring, which has a cone-shaped torroid structure and is capable of being coordinated to a fluorophore. Cyclodextrins improve bioavailability and solubility of the ICG.

In this example of the reporter in reporter matrix 3, the co-ordinated boronic acid derivative and IGC-labeled cyclodextrin is known as a host-guest complex, the ICG (guest) binds with cyclodextrins (host) by occupying the hollow space in the cone structure of the cyclodextrin.

In this example of the reporter in reporter matrix 3, the cyclodextrin protects the ICG from oxidation in biological fluids and prevents dye bleaching in biological fluids, such as blood. This boronic acid-ICG-cyclodextrin complex has poor fluorescence intensity due to shielding of the ICG. But after delivery of the reporter, an affinity reaction causes glucose molecules in the blood stream of the mammal to displace the ICG labeled cyclodextrin from the boronic acid, thereby shifting fluorescence wavelength in response to emitter 4, which is detected by detector 6. In this example of the reporter in reporter matrix 3, the analyte glucose is detected by displacing the ICG labeled cyclodextrin, but any suitable reporter in reporter matrix 3 can be used that is capable of shifting fluorescence wavelength or modulating fluorescence intensity based on the presence of any suitable analyte in the mammal's blood stream.

The fluorescence wavelengths of interest can be detected by detector 6 upon subjecting a portion of the mammal's skin to Near Infrared Spectroscopy (NIRS) with emitter 4. Fluorescence can be detected by detector 6 after a period of time after the reporter matrix 3 is introduced to the mammal's skin and will detect the analyte for another period of time until the reporter matrix 3 has exhausted the stored reporter. This period of time between reporter matrix introduction and detection by detector 6 can be any suitable time, typically about 1 minute to about 6 hours. This is the length of time needed for capillary beds to reach uniform or near uniform diffusion rates and saturation or near saturation conditions.

NIRS allows for deep tissue imaging by exploiting the low absorption of long-wavelength light by skin, bone and blood. This NIRS can be in a wavelength between about 750 nm and 2500 nm, including about 800 nm to about 900 nm. The NIRS signal can come from any suitable type of emitter 4, such as Diode Array spectrometer that uses infrared emitting diodes (IREDs) as sources of near-infrared radiation. The NIRS signal of emitter 4 can also come from Light Emitting Diodes (LED)'s, a laser diode, a light bulb, luciferase, combinations thereof, or any other suitable light source.

Near-infrared radiation emitted from emitter 4 may or may not impinge upon an accompanying optical filter. If the radiation impinges upon an optical filter, each optical filter would be a narrow bandpass filter that passes NIRS radiation at a different wavelength.

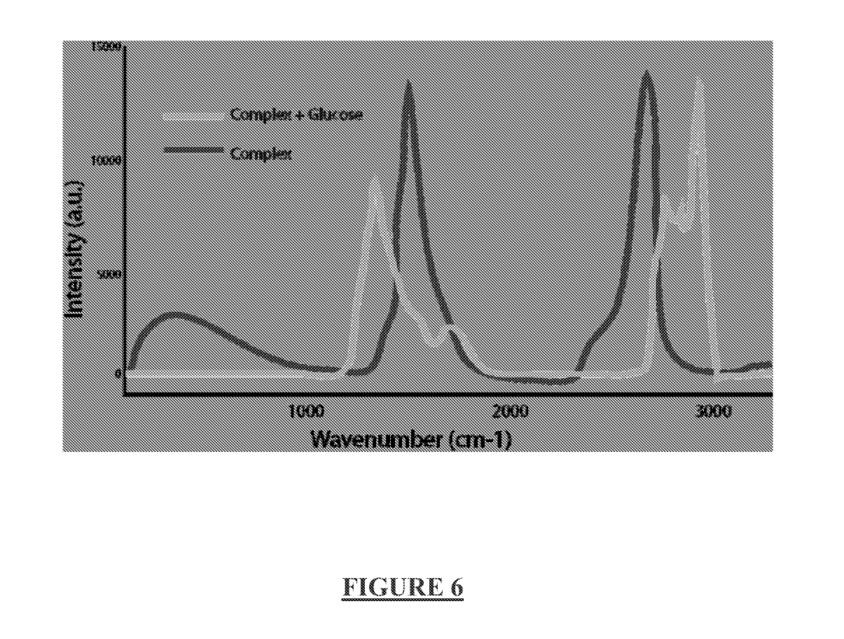

The NIRS from emitter 4 is subjected to a portion of the mammal's skin, passing through the skin a distance into the area below the dermal layers of the mammal. In this example of the reporter in reporter matrix 3, the amount of displaced ICG labeled cyclodextrin emits a fluorescence wavelength that is differentiable from the co-ordinated boronic acid-ICG-cyclodextrin complex. This greater fluorescence is illustrated, as an example, in FIGS. 5 and 6. As an example, in FIG. 5, when the analyte is glucose, and the reporter is a boronic acid cyclodextrin-ICG, there is a difference in intensity (as shown, for example, in FIG. 5b) or wavelength (as shown, for example, in FIG. 5a) between the boronic acid cyclodextrin-ICG itself (shown as “complex”) and the displaced the ICG labeled cyclodextrin (shown as “complex+glucose”). As another example, in FIG. 6 a sample Raman spectra when the analyte is glucose and the reporter is a boronic acid cyclodextrin-ICG, there is a shift in vibration state between the boronic acid cyclodextrin-ICG itself (shown as “complex”) and the displaced the ICG labeled cyclodextrin (shown as “complex+glucose”) in present in two wavenumber ranges.

In the embodiment where analyte binding causes a fluorescent shift, the ratio between unbound and bound reporter correlates to the amount of analyte present in the mammal's blood because the analyte displaces the ICG-cyclodextrin complex and binds itself with the boronic acid derivative. The larger the concentration of the analyte, the more displaced ICG labeled cyclodextrin, the greater the change in fluorescence intensity or the greater the shift in wavelength. For example, if the analyte is glucose, the typical concentration of glucose being detected will be between about 2 mM to about 16 mM. The glucose in this example would then have a corresponding ratio of fluorescence intensities of unbound and bound reporter based on the range of glucose concentration.

In the embodiment where analyte binding causes a change in fluorescent intensity, the ratio between baseline fluorescence will be used to calculate blood-analyte concentration; baseline fluorescence can be measured at a comparable reference area of the body unexposed to the reporter.

The resultant fluorescence intensity of the NIRS radiation is measured and detected by detector 6. The detector 6 can be a suitable fluorescence detector, including but not limited to a silicon photodetector, a photodiode having an interference filter, a prism or grating having a charge-coupled device array detection element, a photomultiplier tube, and combinations thereof.

Based on the fluorescence emission intensity of wavelengths of interest detected by detector 6, which are dictated by type of fluorophore and reporter components a calculation can be made to determine the concentration of the analyte in the blood stream, as described above.

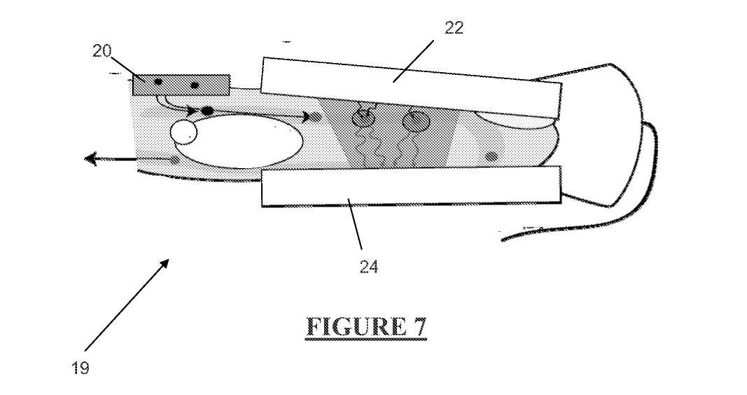

Another embodiment of the present system includes a patch that is separated from an emitter and detector. In FIG. 7, system 19 includes a separate patch 20, separate emitter 22 and separate detector 24. Separate patch 20 operates in a fashion similar to patch 2 and reporter matrix 3, in that separate patch 20 can be any suitable patch capable of transmitting an optical reporter through a mammal's skin for a period of time that may include an adhesive. Separate patch 20 transmits the optical reporter through the mammal's skin in one location, while separate emitter 22 emits radiation in another location that is downstream in the blood stream of the mammal as compared to the separate patch. Separate emitter 22 can be any suitable source that is capable of delivering near-infrared radiation. Separate detector 24 can be any suitable detector that is capable of detecting NIRS radiation.

As one example, and as shown in FIG. 7, system 19 can be placed around the mammal's finger, with the separate emitter 22 and separate detector 24 being electronically connected to each other and transmit information through a wire (as shown in FIG. 5) or through a wireless communication.

A limitation of previous methods of analyte testing is the inability to assess the temporal dynamic of that analyte. Any of the present systems are able to transmit the signal received from detector 6, through transmitter 10, or from separate detector 24 to a series of outputs to a receiver once every set time period. This time period can be any period suitable for monitoring for the varying levels of a specific analyte, including but not limited to between 1 second and 24 hours. For example, if the analyte is glucose, a person may want to continuously monitor their glucose levels throughout the day, therefore the time period would be about every one minute to every half hour. If the analyte were something other than glucose, such as iron levels, a longer period between measurements may be desired.

The transmitter 10, can encrypt the signal received from detector 6 and transmit the signal as a Health Insurance Portability and Accountability Act of 1996 (HIPPA) compliant encrypted signal to an external analysis receiver. The external analysis device can then determine the analyte level and can also, optionally, transmit the determined analyte level to a unit capable of delivering a chemical in response to the analyte level. One example of this unit would be an insulin pump that would be capable of delivering insulin based on received outputs.

Although the above example describes an insulin pump, the unit capable of delivering a chemical can deliver any suitable chemical to the mammal in response to output received from transmitter 10.

Any of the present systems are also capable of delivering an alarm based on output of the transmitter 10. This alarm can be a component of the patch 2 or it can be a component of the receiver. The alarm itself can be an auditory signal and/or a visual signal and provide notification that the level of analyte requires attention, such as when the level of analyte is too high or too low.

The reporter of any of the present systems can be delivered continuously or nearly continuously delivered to ensure near uniform concentration within a local capillary bed in the area of any of the present patches. FIG. 8 is a series of in situ transdermal permeation tests in pig skin, with 0%, 5%, and 10% enhancement with oleic acid. As shown in FIG. 8, the transdermal diffusion capability of the reporter is shown within pig skin, and is shown to be uniform or nearly uniform in density along the width of the sample and in density along the depth of the sample. As shown in FIG. 8, deep transdermal permeation is demonstrated in all cases. FIG. 8 shows reporter permeation with chemical enhancement; in alternative embodiments of the application, physical enhancements are used, such as ultrasound, heat, and microneedles. Furthermore, no enhancement may be necessary to achieve sufficient flux of reporter into the bloodstream.

### Example 1

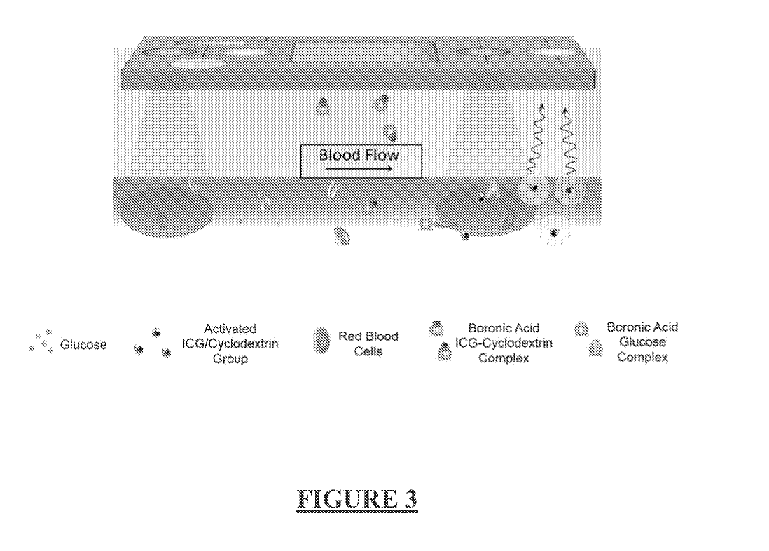

In the following example and in FIGS. 2 and 3, a matrix-type transdermal patch 2 will be used for assessing the glucose levels of a human. This matrix-type patch 2 includes a backing layer (not shown), a reporter matrix 3 and a liner (not shown), the patch able to release a near constant dosage of reporter for extended periods of up to seven days with minimal, if any skin irritation. A boronic acid derivative co-ordinated to an Indocyanine green (ICG)-labeled cyclodextrin is present in reporter matrix 3 and is a glucose specific reporter. ICG-labeled cyclodextrin displays optimal blood borne-fluorescence in vivo at about 0.08 mg/L, so assuming a network of 10 capillaries, each 10 μm diameter, within a 1 cm² region and blood flow rate of 10 mL/min, a steady-state reporter delivery of about 0.5 mg/h will be delivered from reporter matrix 3.

Also included in the patch will be one or more of Near Infrared (NIR) Light Emitting Diodes (LEDs) 4 and detector 6 capable of detecting NIR signals. Although FIG. 2 indicates that there is one emitter 4 and one detector 6, a plurality of emitter and detectors can be included. The emitter 4 and detector 6 will be integrated with a microprocessor 8, transmitter 10 and a power supply 12.

In blood excited with 800 nm light by emitter 4, the quantum yield of free ICG present at 0.08 mg/L is about 0.013, with an emission wavelength of about 830 nm. Exciting ICG at this concentration, in a 10 μm by 10 μm section of capillary, at a depth of 5 mm using 1 mW of 800 nm light from emitter 4, will result in about 30 μW of 830 nm light being incident on the detector 6, producing a signal of about 1 mV. The power incident on the detector 6 will be above the noise floor of the device, which is 10 pW.

Penetration depth of the emission from emitter 4 is proportional to one-third of the spacing between the emitter 4 and the detector 6; to allow for a detection depth of approximately 1 cm, in this example, there will be a three cm space between the emitter 4 and the detector 6. Once the detector 6 has detected the fluorescence signal from bound and unbound reporter complex, the signal can be encrypted as a Health Insurance Portability and Accountability Act of 1996 (HIPPA) compliant encrypted signal by processor 8 and transmitted to an external analysis device by transmitter 10.

A receiver (not shown) receives the signal from transmitter 10 and has software to calculate absolute glucose levels based on calibration parameters, a reference NIR signal created by reference emitter 14 and reference detector 16 (to account for variation in hydration, temperature, movement and hematocrit) and a patient-specific transfer function to account for differences in tissues and capillary bed depths/geometries.

The reference emitter 14 and reference detector 16, as shown in FIG. 3, can be used to determine the base level of fluorescence in the person's blood stream prior to application of the reporter. In this example, the patch 2 is arranged so that reference emitter 14 and reference detector 16 are placed above stream of reporter matrix 3. Since reference emitter 14 and reference detector 16 are above stream of reporter matrix 3, little to no reporter will be present and the background fluorescence of the person's blood can be detected by reference detector 16. The background fluorescence of the person's blood can then be subtracted from the fluorescence detected by detector 6 to determine the absolute levels of fluorescence caused by glucose and reporter in bound and unbound states

Based on the measured glucose levels, the patch can be adapted to contact an insulin pump to deliver insulin if the measured glucose levels are at or above a predetermined threshold. In this example, if the person's measured glucose levels reach hyperglycemic levels (>120 mg/dL), the insulin pump is contacted to deliver a dosage of insulin proportional to the glucose levels. Additionally, if the person's measured glucose levels reach 180 mg/dL or greater, the patch will create an audible or visual alarm to notify the person that their glucose levels are too high. Similarly, if the person's measured glucose levels reach 60 mg/dL or less, which is considered hypoglycemia and can lead to syncope or diabetic coma, an alarm will notify the person.

Over time, the measured glucose levels can be used to develop an adaptive algorithm that is based on the specific person. Because glucose levels are part of a regulated control system, levels are the product of an input, a process and a feedback-namely, food intake, absorption of glucose into the blood and release of insulin following activation of beta cells in the islets of Langerhans of the pancreas, individual adaptive algorithms can be developed. These algorithms can be useful for predicting hypo- and hyperglycemic events and will assist people in the prediction of their glycemic responses to means and activities. These adaptive algorithms will likely be based on Bergmans Minimal Model.

### Example 2

In the following example, absorbance data for several reporters is discussed. In the following figures β-cyclodextrins contribute to an enhanced absorption signal of indocyanine green (ICG) in the near infrared region. Higher concentrations of ICG indicate an aggregation-based peak at about 715 nm that is reduced by addition of cyclodextrin. Guest-host complexation between ICG and cyclodextrins may prevent aggregation of ICG in solution.

The absorption spectra of ICG and several ICG-β-cyclodextrin solutions of 1 μM, 10 μM and 50 μM in aqueous solution was measured. For all complexation experiments, ICG solutions were added to β-cyclodextrin solutions to arrive at final β-cyclodextrin concentrations of 8 mM. During this time, samples were kept at room temperature, about 70° F., and exposed to minimal amounts of light. All measurements were performed in duplicate using an Evolution 300 UV/Vis spectrophotometer from ThermoFisher Scientific.

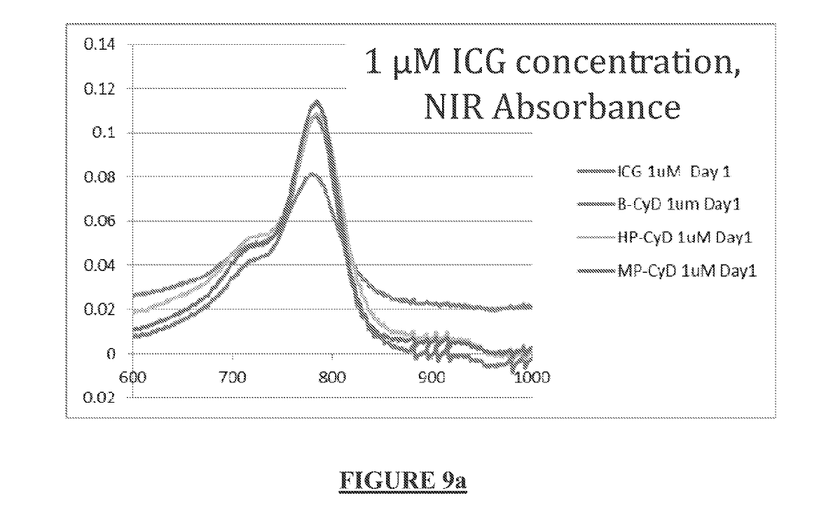

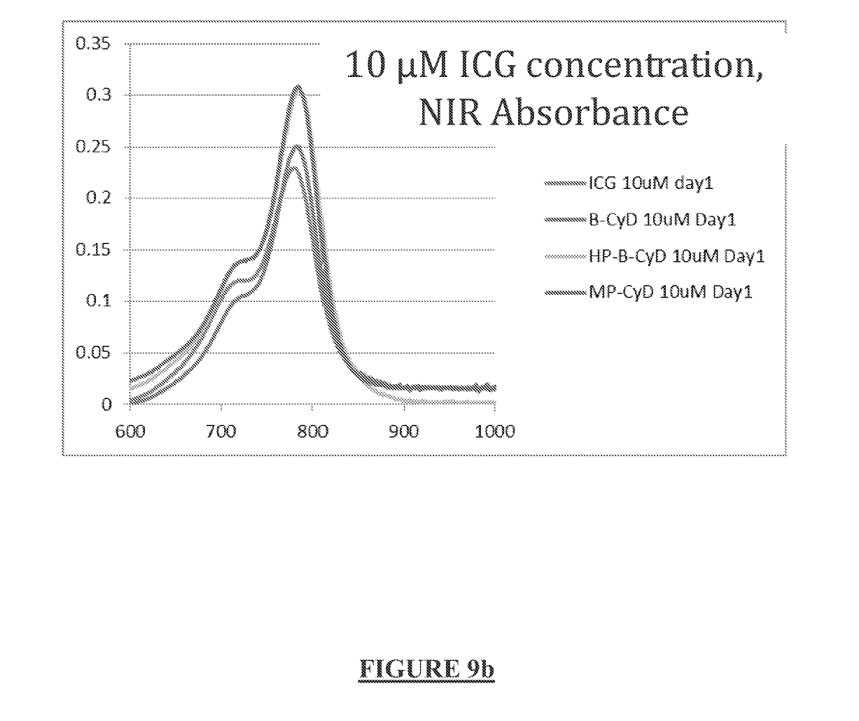

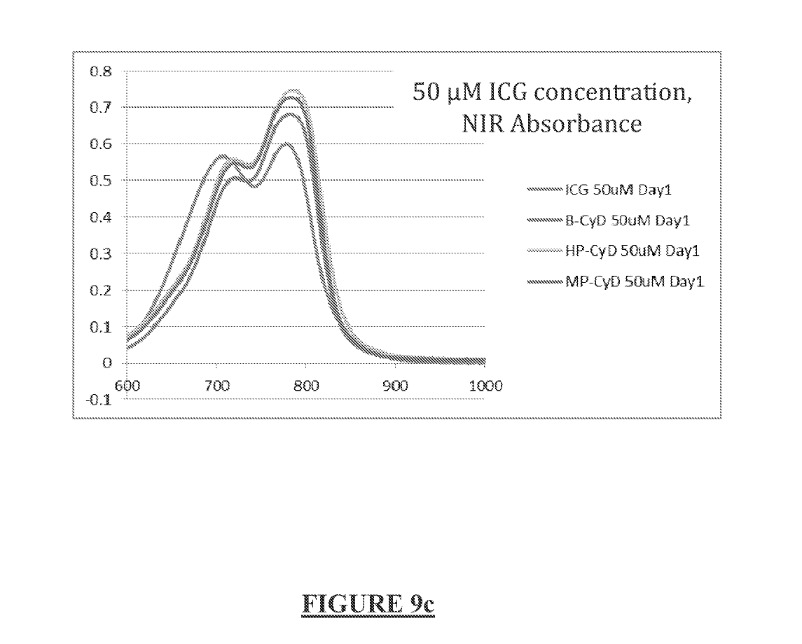

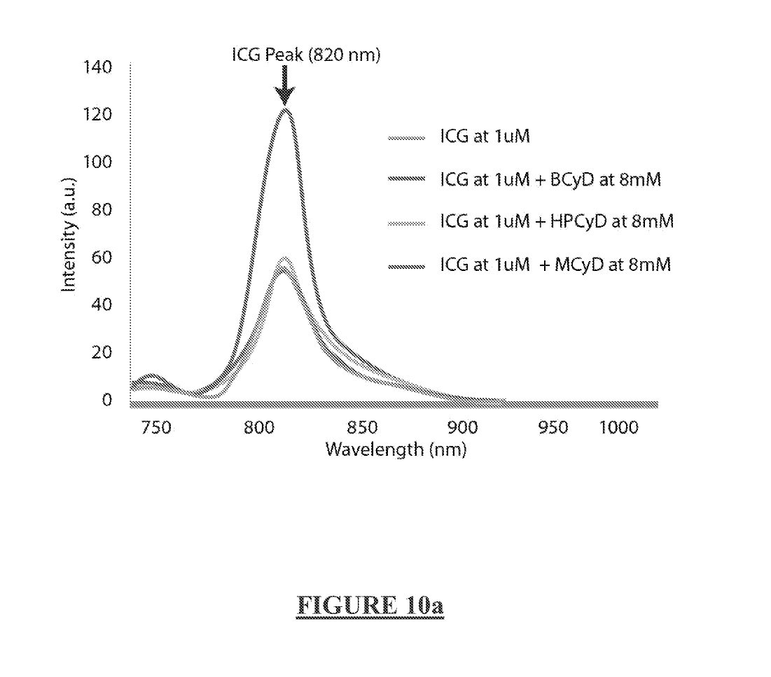

As can be seen in FIGS. 9a-9c, which illustrate the Near Infrared (NIR) absorbance of different ICG concentration, varying cyclodextrin compounds absorb to varying degrees. In FIGS. 9a-9c, four compounds were analyzed, including an ICG cyclodextrin complex, an ICG β-cyclodextrin complex, an ICG hydroxypropyl-β-cyclodextrin complex and an ICG methyl-β-cyclodextrin complex.

As can be seen in FIGS. 9a and 9b, ICG at 1 μM and 10 μM has enhanced absorbance when included in a solution of 8 mM β-cyclodextrin complex, hydroxypropyl-β-cyclodextrin complex or methyl-β-cyclodextrin complex. As can be seen from FIG. 9c, ICG at 50 μM has enhanced absorbance at decreased aggregation, which is the ratio between peaks at about 700 nm and about 780 nm, where the peak at about 700 nm represents aggregated ICG, when included in a solution of 8 mM β-cyclodextrin complex, hydroxypropyl-β-cyclodextrin complex or methyl-β-cyclodextrin complex.

### Example 3

In the following example, fluorescence data for several reporters are discussed. In the following figures, the effect of β-cyclodextrin, hydroxypropyl β-cyclodextrin and methyl β-cyclodextrin on the near infrared fluorescence (NIRF) of indocyanine green (ICG) is demonstrated. For each formulation, cyclodextrin concentration was kept constant at 8 mM, resulting in an excess of free cyclodextrin in the solution. The addition of boronic acid to the complex was tested via a freeze dry technique and whether this technique would enable fluorescent glucose detection.

As noted above, the addition of boronic acid was via a freeze dry technique, under this freeze drying method the complexation of ICG with various cyclodextrins can be achieved by stirring aqueous solutions of ICG and cyclodextrins for about 24 hours or more, and then lyophilizing the solution to obtain dry powders.

Another technique for preparing ICG complexes is to first prepare an ICG stock solution at a concentration of 1 mM/L of deionized water. β-cyclodextrin stock solutions (10 mM concentration) were prepared by stirring β-cyclodextrin powders in deionized for two hours or more to dissolve the powders. After that, aliquots of the stock solution of ICG (1 mM/L) were added to 8 mL aliquot of the cyclodextrins stock solutions to obtain three final dye concentrations of 1, 10 and 50 μM with a constant concentration of 8 mM for the cyclodextrins. The resulting 10 mL solution was shaken for a few minutes, resulting in a complex solution.

Under the freeze drying technique, fluorescence testing and optimization of the complexes were performed at the Ultrafast Optics Lab at Brookhaven National Laboratory (Brookhaven, N.Y.). A two-sided polished 1-mm thickness NIR cuvette and a SC450PP Supercontinuum Laser (Fianium, Southhampton, United Kingdom) were used. Excitation was centered at 633+/−30 nm, with 100 μW excitation intensity at 20 MHZ repetition rate. After filtering and focusing, emission light was long-pass filtered at 640+/−1 nm and detected by a liquid N₂ cooled, deep depleted, back-illuminated CCD (JY Horiba, Edison, N.J.). Data collection was doe with SynerJY software (JY Horiba, Edison, N.J.).

Cuvettes were washed with 95% sulfuric acid between scans. All scans were performed in duplicate, visually inspected and averaged. As an additional measure, the Raman vibrational spectra for the complex was examined before and after addition of glucose. The excitation wavelength was 530 nm, with an integration window of five seconds. Raman displays a specific spectrum for each compound, this spectrum is characteristic of the vibrational state of the compound. FIG. 6 illustrates the Raman spectra of the reporter before and after the addition of 8 mM of glucose. A significant shift in both peaks is observed.

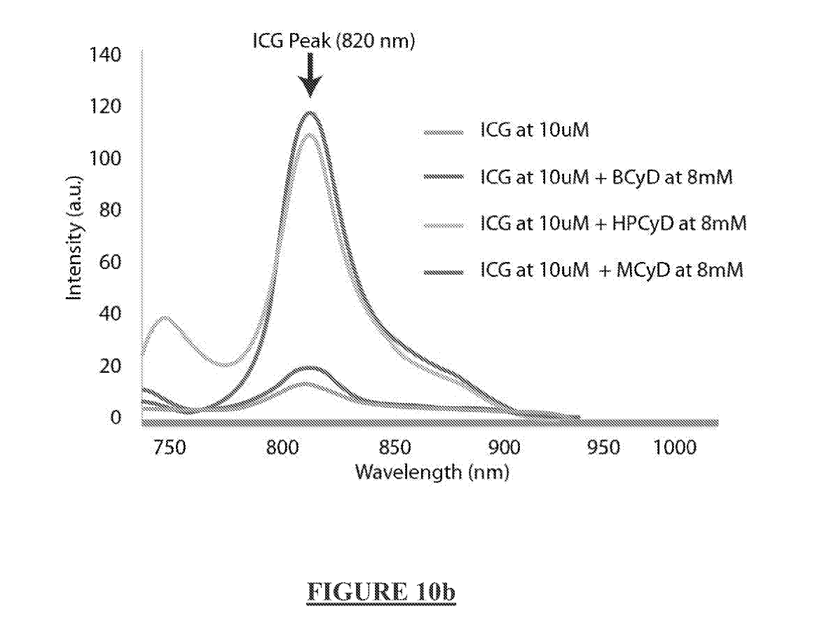

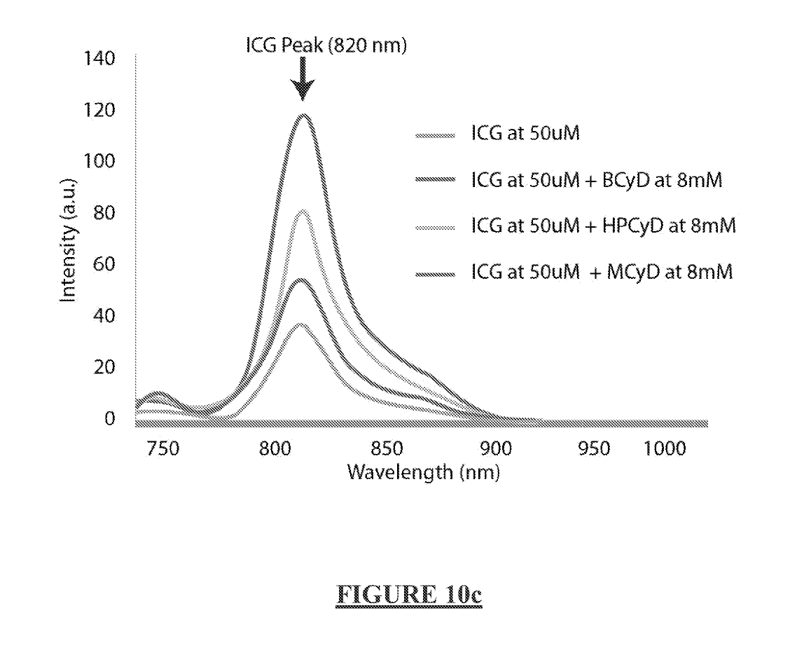

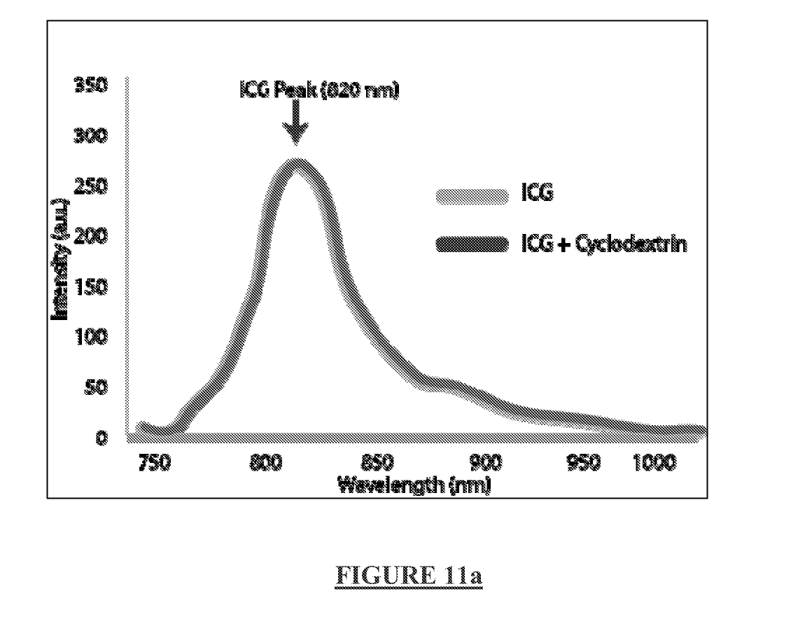

As can be seen in FIGS. 10a-10c, which illustrate the fluorescence of different ICG concentration, varying cyclodextrin compounds fluoresce to varying degrees. In FIGS. 10a-10c, four compounds were analyzed, including an ICG cyclodextrin complex, an ICG β-cyclodextrin complex, an ICG hydroxypropyl-β-cyclodextrin complex and an ICG methyl-β-cyclodextrin complex. These complexes can also be complexed with boronic acid.

As can be seen in FIGS. 10a-10c, varying compounds, at varying concentrations, fluoresce to differing levels at about 805 nm.

### Example 4

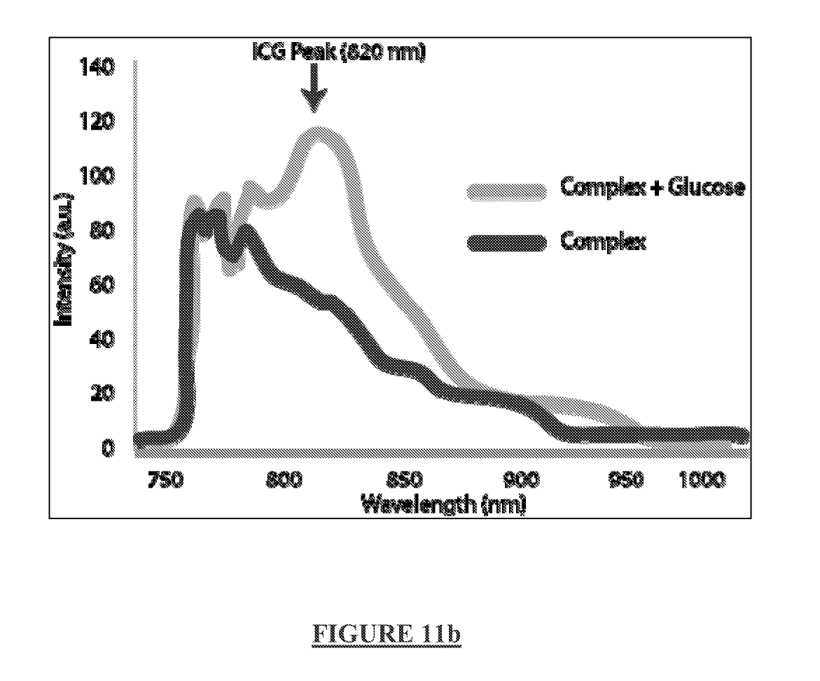

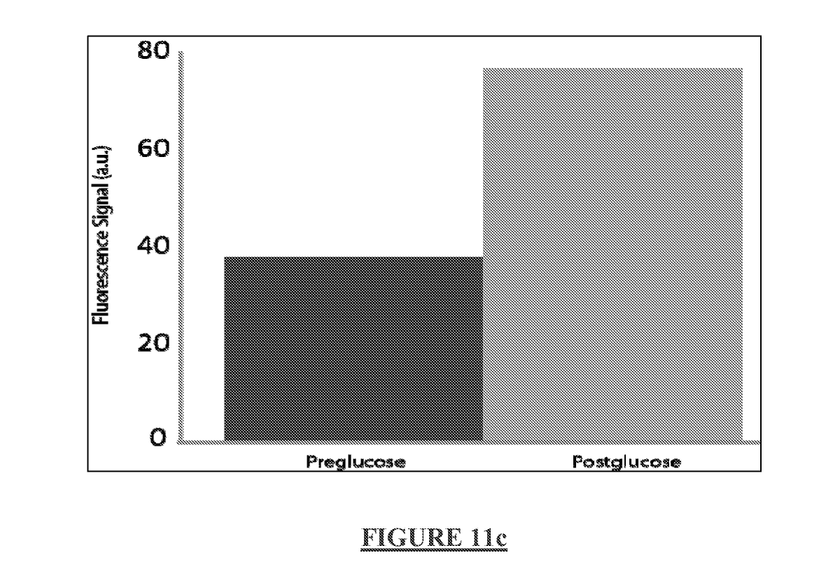

FIGS. 11a-11c illustrate the ability of the complexes described herein to be sensed with Near Infrared Spectroscopy (NIRS) non-invasively. As can be seen in FIG. 11a, ICG and a complex of ICG and β-cyclodextrin exhibit fluorescence peaks at about 820 nm. As can be seen in FIG. 11b, a complex of ICG, β-cyclodextrin and boronic acid exhibits quenched fluorescence and in the presence of glucose, this complex exhibits an increase in fluorescence. As can be seen in FIG. 11c, which illustrates integrated fluorescence over the 810-830 nm region, fluorescence in this region passes through tissues relatively unscattered.

The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

What is claimed is:

1. A method for determining the concentration of an analyte in a mammal's bloodstream, the method comprising:
applying a patch to the mammal's skin, the patch configured to transmit a reporter through the mammal's skin, wherein the reporter is configured to have an affinity for the analyte and is capable of exhibiting a shift in fluorescence upon binding to the analyte;
subjecting a portion of the mammal's skin to Near Infrared Spectroscopy (NIRS);
detecting fluorescence emission spectrum; and
calculating the concentration of blood analyte based on measured fluorescence.

2. The method of claim 1, wherein the reporter comprises a boronic acid derivative and a non-specific fluorophore bound to a cyclic chain-sugar molecule.

3. The method of claim 2, wherein the reporter comprises a boronic acid derivative co-ordinated with at least one of an indocyanine green-labeled cyclodextrin complex, an ICG β-cyclodextrin complex, an ICG hydroxypropyl-β-cyclodextrin complex and an ICG methyl-β-cyclodextrin complex.

4. The method of claim 2, wherein the analyte binds with the boronic acid derivative, displacing the non-specific fluorophore bound to the sugar molecule.

5. The method of claim 1, further comprising a step of monitoring the concentration of the analyte over a period of time.

6. The method of claim 1, wherein the analyte comprises glucose, insulin, hormones, drugs and combinations thereof.

7. The method of claim 6, wherein the analyte is glucose.

8. The method of claim 1, wherein the patch delivers the reporter for up to 7 days.

9. The method of claim 1, wherein a unit delivers a substance in response to concentration of the analyte.

10. The method of claim 9, wherein the unit is an insulin pump.

11. The method of claim 1, wherein the step of detecting fluorescence emission spectrum allows calculation of glucose concentration, [glucose], by solving the equilibrium equation:
k=[reporter][glucose]/[reporter+glucose]
wherein k is an empirically derived binding constant between the reporter complex and glucose in blood, and [reporter] and [reporter+glucose] is a ratio acquired from measuring fluorescence at two wavelengths, representing the unbound and bound states of said reporter.

12. The method of claim 1, wherein the shift in fluorescence is a shift in fluorescence wavelength

13. The method of claim 1, wherein the shift in fluorescence is a modulation in fluorescence intensity.

14. An analyte monitoring system, the system comprising:
a patch containing a reporter that is configured to be transmitted through a mammal's skin,
a NIRS emitter and a NIRS detector operably connected to a processor, a power supply and a transmitter; and
a receiver capable of receiving information from the transmitter.

15. The analyte monitoring system of claim 14, wherein the NIRS emitter, the NIRS detector, the processor and the transmitter are integrated in the patch.

16. The analyte monitoring system of claim 14, wherein the analyte concentration is transmitted once every time period, wherein the time period is from about 1 second to about 24 hours.

17. The analyte monitoring system of claim 14, wherein the reporter comprises a boronic acid derivative and a non-specific fluorophore bound to a cyclic ring-sugar molecule.

18. The analyte monitoring system of claim 14, wherein the reporter comprises a boronic acid derivative co-ordinated with at least one of an indocyanine green-labeled cyclodextrin complex, an ICG β-cyclodextrin complex, an ICG hydroxypropyl-β-cyclodextrin complex and an ICG methyl-β-cyclodextrin complex.

19. The analyte monitoring system of claim 14, wherein the analyte comprises glucose, insulin, hormones, drugs and combinations thereof.

20. The analyte monitoring system of claim 19, wherein the analyte is glucose.
